(19) United States
(12) Patent Application Publication
(10) Pub. No.: US 2005/0147588 A1
(43) Pub. Date: Jul. 7, 2005

(54) Methods for treatment of obesity and for promotion of weight loss

(76) Inventors: Chih-Ping Liu, San Francisco, CA (US); Henry W. Lopez, Napa, CA (US)

Correspondence Address: PERKINS COIE LLP, P.O. BOX 2168, MENLO PARK, CA, 94026, US

(21) Appl. No.: 10/991,653

(22) Filed: Nov. 17, 2004

Related U.S. Application Data
(60) Provisional application No. 60/523,077, filed on Nov. 17, 2003.
(60) Provisional application No. 60/532,851, filed on Dec. 24, 2003.

(51) Int. Cl. A61K 9/68; A61K 38/21

(52) U.S. Cl. 424/85.4; 424/48

(57) ABSTRACT

Methods for promoting weight loss and/or preventing weight gain, particularly excessive weight gain, in subjects carrying excess body weight are described. The methods involve administering interferon-tau to the subject. Also described are methods for reducing body weight or preventing weight gain in subjects at risk of developing or already suffering from conditions caused by or exacerbated by excess body weight, such as diabetes, and particularly autoimmune diseases, such as rheumatoid arthritis and multiple sclerosis, and cardiovascular conditions, such as hypertension.

This application claims the benefit of U.S. Provisional Application No. 60/523,077 filed Nov. 17, 2003, and to U.S. Provisional Application No. 60/532,851 filed Dec. 24, 2003. Both documents are incorporated by reference herein in their entirety.

### FIELD OF THE INVENTION

The present invention is directed to the fields of obesity, causing weight loss, preventing weight gain, and to treatment of conditions associated with, exacerbated by, or directly caused by obesity. More specifically, the invention relates to methods of promoting weight loss, preventing weight gain, and of treating conditions associated with, exacerbated by, or directly caused by the state of being overweight or obese, by administering interferon-tau.

### BACKGROUND OF THE INVENTION

Obesity involves an excessive accumulation of body fat and is widely considered to be a major public health problem, associated with substantially increased morbidity and mortality, as well as psychological problems, reduced economic achievement, and discrimination. Obesity is the second leading cause of preventable death in the U.S. and currently more than half of the adult population is overweight and almost one quarter of the population is considered obese (a body mass index (BMI) greater than or equal to 30). While obesity alone is a serious health concern, it also is known to contribute, cause or exacerbate other health problems. These problems include coronary heart disease, stroke, obstructive sleep apnea, diabetes mellitus, gout, hyperlipidemia, osteoarthritis, reduced fertility, impaired psychosocial function, reduced physical agility and increased risk of accidents, and impaired obstetrical performance.

Causes of obesity remain unclear. However, whether obesity is of genetic origin or is promoted by a genotype-environment interaction, or both, it is evident that energy intake must have exceeded metabolic and physical (work) energy expenditure for there to have been surplus energy available for fat deposition. Considerable uncertainty remains concerning the relative importance of different mechanisms in achieving this positive energy balance.

Treatment of obesity is difficult. Although it is well-established that morbidity and mortality are increased in obese individuals, it is unclear whether dieting results in decreased long-term risk of early death. The major obesity intervention has been the many different forms of dieting, which are often fads without a sound scientific basis. A further important obesity intervention is physical activity which increases energy expenditure, both during the actual period of exercise and during the subsequent period of rest. Another method of coping with obesity is via therapeutic aids, and drugs currently approved by the FDA for the treatment of obesity include phentermine, fenfluramine, sibutramine, orlistat, and phenylpropanolamine. Side effects occur with all these drugs. For example, the administration of fenfluramine and phentermine for the treatment of obesity resulted in cardiac valve damage in some patients and ultimately led to the withdrawal of fenfluramine from the market. Sibutramine increases blood pressure in a subset of patients, and orlistat may have unpleasant gastrointestinal side effects.

It is therefore evident that obesity is a problem, and that no reliable treatment thereof has been established. There is a continuing need to develop drugs and treatment regimes effective in the alleviation of obesity.

### SUMMARY OF THE INVENTION

Accordingly, in one aspect, the invention provides a method for promoting weight loss in a human subject.

In another aspect, the invention provides a method of preventing weight gain in a human subject.

In yet another aspect, the invention provides a method of preventing excessive weight gain in a human subject.

In still another aspect, the invention provides a method to treat patients having a body mass index sufficient for categorization of “overweight”, more preferably sufficient for categorization of “obese” and suffering from a secondary condition caused by, exaceberated by, or related to being overweight or obese.

In yet another aspect, the invention provides a method for preventing weight gain or for promoting weight loss in persons having a body mass index sufficient for categorization of “overweight”, more preferably sufficient for categorization of “obese”, and at risk of developing a medical condition caused by or related to being overweight or obese.

In a further aspect, the invention provides a method to prevent excess weight gain in persons who are at a developmental stage where a certain amount of weight gain is normal and expected.

In another aspect, the invention provides a method for treating a patient population having a body mass index greater than about 25 and suffering from an autoimmune disorder or a cardiovascular disorder. Treatment of the population and of persons within the population is contemplated.

These and other objects and features of the invention will be more fully appreciated when the following detailed description of the invention is read in conjunction with the accompanying drawings.

### BRIEF DESCRIPTION OF THE DRAWINGS

FIG. 1A is a graph of body weight of mice, in grams, as a function of days for mice fed a high fat, high sucrose diet (squares), a high fat, high sucrose diet plus interferon-tau (IFNτ) via oral gavage (diamonds), or a conventional rodent chow (control, triangles);

FIG. 1B is a bar graph showing the weight of fat depots excised from mice treated for 14 days as described in FIG. 1A, the fat depots excised from subcutaneous inguinal, gonadal, retroperitonial, and mesenteric tissues;

FIG. 2A is a graph of body weight of mice, in grams, as a function of days, where for days 1-23 mice were fed a high fat diet, and beginning on day 24 were additionally given via oral gavage a daily dose of 1 μg IFNτ (squares) or 10 μg IFNτ (triangles) or phosphate buffered saline (control, circles);

FIG. 2B is a bar graph showing the average body weight of mice, in grams, of three treatment groups, where mice in all treatment groups were fed a high fat diet for 54 days, where beginning on day 24 the mice were additionally given via oral gavage a daily dose of 1 μg IFNτ or 10 μg IFNτ or phosphate buffered saline (control), the dotted bars showing the average mouse body weight for each treatment group after 54 days, and the stripped bars showing the increase in body weight during the treatment period of days 24-54;

FIG. 3 is a graph of blood glucose concentration in mice, in mg/dL, as a function of time, in minutes, where on day 40 of the 54 day described in FIG. 2A the mice in each treatment group (control (circles), 1 μg IFNτ squares), 10 μg IFNτ (triangles)) were fasted for 4 hours and given a glucose load of 2 g/kg body weight and blood samples drawn at defined intervals for glucose clearance measurement;

FIG. 4 is a graph of blood glucose concentration in mice, in mg/dL, as a function of time, in minutes, where on day 44 of the 54 day described in FIG. 2A the mice in each treatment group (control (circles), 1 μg IFNτ (squares), 10 μg IFNτ (triangles)) were fasted for 4 hours and an intraperitoneal insulin suppression test was done by giving an insulin load of 0.75 units/kg body weight and blood samples drawn at defined intervals for glucose clearance measurement;

FIG. 5 is a bar graph showing average weight, in grams, of fat depots excised from inguinal, gonadal, retroperitoneal, mesenteric regions of mice at the completion of the 54 day study described in FIG. 2A for the mice in each treatment group of control (dotted bars), 1 μg IFNτ (cross hatched bars), 10 μg IFNτ (vertical stripes);

FIG. 6A shows average, cumulative food consumption, in grams, as a function of time, in days, by the mice in each treatment group (control (circles), 1 μg IFNτ (squares), 10 μg IFNτ (triangles)) in the study described in FIG. 2A; and

FIG. 6B shows the average, daily food consumption, in grams, as a function of time grouped into 4-5 day test periods, by the mice in each treatment group (control (circles), 1 μg IFNτ (squares), 10 μg IFNτ (triangles)) in the study described in FIG. 2A.

### BRIEF DESCRIPTION OF THE SEQUENCES

SEQ ID NO:1 is the nucleotide sequence of a synthetic gene encoding ovine interferon-T (IFNτ).

SEQ ID NO:2 corresponds to an amino acid sequence of mature ovine interferon-τ(IFNτ; oTP-1; GenBank Accession No. Y00287; PID g1358).

SEQ ID NO:3 corresponds to an amino acid sequence of mature ovine IFNτ, where the amino acid residues at positions 5 and 6 of the sequence are modified relative to the sequence of SEQ ID NO:2.

SEQ ID NO:4 is a synthetic nucleotide sequence encoding the protein of SEQ ID NO:3.

### DETAILED DESCRIPTION OF THE INVENTION

I. Definitions

Interferon-tau, abbreviated as IFNτ or interferon-1, refers to any one of a family of interferon proteins having at least one characteristic from each of the following two groups of characteristics: (i) (a) anti-luteolytic properties, (b) anti-viral properties, (c) anti-cellular proliferation properties; and (ii) about 45 to 68% amino acid homology with α-Interferons and greater than 70% amino acid homology to known IFNτ sequences (e.g., Ott, et al., J. Interferon Res., 11: 357 (1991); Helmer, et al., J. Reprod. Fert., 79: 83 (1987); Imakawa, et al., Mol. Endocrinol, 3: 127 (1989); Whaley, et al., J. Biol. Chem., 269: 10846 (1994); Bazer, et al., WO 94/10313 (1994)). Amino acid homology can be determined using, for example, the LALIGN program with default parameters. This program is found in the FASTA version 1.7 suite of sequence comparison programs (Pearson and Lipman, PNAS, 85: 2444 (1988); Pearson, Methods in Enzymology, 183: 63 (1990); program available from William R. Pearson, Department of Biological Chemistry, Box 440, Jordan Hall, Charlottesville, Va.). IFNτ sequences have been identified in various ruminant species, including but not limited to, cow (Bovine sp., Helmer, S. D., J. Reprod. Fert., 79: 83 (1987); Imakawa, K., Mol. Endocrinol., 119: 532 (1988)), sheep (Ovine sp.), musk ox (Ovibos sp.), giraffe (Giraffa sp., GenBank Accession no. U55050), horse (Equus caballus), zebra (Equus burchelli, GenBank Accession no. NC005027), hippopotamus (Hippopotamus sp.), elephant (Loxodonta sp.), llama (Llama glama), goat (Capra sp., GenBank Accession nos. AY357336, AY357335, AY347334, AY357333, AY357332, AY357331, AY357330, AY357329, AY357328, AY357327), and deer (Cervidae sp.). The nucleotide sequences of IFNτ for many of these species are reported in public databases and/or in the literature (see, for example, Roberts, R. M. et al., J. Interferon and Cytokine Res., 18: 805 (1998), Leaman D. W. et al., J. Interferon Res., 12: 1 (1993), Ryan, A. M. et al., Anim. Genet., 34: 9 (1996)). The term “interferon-tau” intends to encompass the interferon-tau protein from any ruminant species, exemplified by those recited above, that has at least one characteristic from each of the following two groups of characteristics listed above.

Ovine IFNτ (IFNτ) refers to a protein having the amino acid sequence as identified herein as SEQ ID NO:2, and to proteins having amino acid substitutions and alterations such as neutral amino acid substitutions that do not significantly affect the activity of the protein, such as the IFNα protein identified herein as SEQ ID NO:3. More generally, an ovine IFNα protein is one having about 80%, more preferably 90%, sequence homology to the sequence identified as SEQ ID NO:2. Sequence homology is determined, for example, by a strict amino acid comparison or using one of the many programs commercially available.

Treating a condition refers to administering a therapeutic substance effective to reduce the symptoms of the condition and/or lessen the severity of the condition.

Oral refers to any route that involves administration by the mouth or direct administration into the stomach or intestines, including gastric administration.

Intestine refers to the portion of the digestive tract that extends from the lower opening of the stomach to the anus, composed of the small intestine (duodenum, jejunum, and ileum) and the large intestine (ascending colon, transverse colon, descending colon, sigmoid colon, and rectum).

The terms treating obesity and treatment of obesity, as used herein, include prophylaxis as well as alleviation of established obesity. In addition to the treatment of obesity, the terms contemplate treatment of conditions associated with obesity and conditions exacerbated by the state of being obese.

II. Methods of Weight Reduction and Weight Management

In a first aspect, the invention contemplates methods for promoting weight loss, for preventing weight gain, and for preventing excessive weight gain by administering IFNτ. As will be discussed below, administration of INFτ to overweight subjects resulted in a reduction of fat deposition. Further, administration of INFτ to subjects on a high fat diet had less weight gain and fat deposition than subjects fed the same diet but with no delivery of IFNτ. In the sections below, IFNτ is described and studies establishing the protein's utility in the claimed methods are discussed.

A. IFNτ

Interferon-tau (hereinafter “IFNτ” or “interferon-τ”) was discovered originally as a pregnancy recognition hormone produced by the trophectoderm of ruminant conceptuses (Imakawa, K. et al, Nature, 330: 377-379, (1987); Bazer, F. W. and Johnson, H. M., Am. J. Repro. Immunol., 26: 19-22, (1991)). The distribution of the IFNτ gene is restricted to ruminants, including cattle, sheep, and goats, (Alexenko, A. P. et al., J. Interferon and Cytokine Res., 19: 1335-1341, (1999)) but has been shown to have activity in cells belonging to other species including humans and mice (Pontzer, C. H. et al., Cancer Res., 51: 5304-5307, (1991); Alexenko, A. P. et al., J. Interferon and Cytokine Res., 20: 817-822, (2000)). For example, IFNα has been demonstrated to possess antiviral, (Pontzer, C. H. et al., Biochem. Biophys. Res. Commun., 152: 801-807, (1988)), antiproliferative, (Pontzer, C. H., et al., 1991) and immunoregulatory activities (Assal-Meliani, A., Am. J. Repro. Immunol., 33: 267-275 (1995)).

While IFNτ displays many of the activities classically associated with type I IFNs, such as interferon-α and inteferon-β, considerable differences exist between IFNτ and the other type I IFNs. The most prominent difference is the role of IFNτ in pregnancy in ruminant species. The other IFNs have no similar activity in pregnancy recognition. Also different is viral induction. All type I IFNs, except IFNτ, are induced readily by virus and dsRNA (Roberts, et al., Endocrine Reviews, 13: 432 (1992)). Induced IFN-α and IFN-β expression is transient, lasting approximately a few hours. In contrast, IFNτ synthesis, once induced, is maintained over a period of days (Godkin, et al., J. Reprod. Fert., 65: 141 (1982)). On a per-cell basis, 300-fold more IFNτ is produced than other type I IFNs (Cross, J. C. and Roberts, R. M., Proc. Natl. Acad. Sci. USA 88: 3817-3821 (1991)).

Another difference lies in the amino acid sequences of IFN-τ and other type I interferons. The percent amino acid sequence similarity between the interferons α_{2b}, ω₁, γ, and τ are summarized in the table below.

| | rHuIFNα_{2b} | rHuIFNβ₁ | rHuIFN₁ω₁ | rHuIFN_{γ} | rOvIFNτ |
|---|---|---|---|---|---|
| RhuIFNα_{2b} | | 33.1 | 60.8 | 11.6 | 48.8 |
| RhuIFNβ₁ | 33.1 | | 33.1 | 12.2 | 33.8 |
| RhuIFNω₁ | 60.8 | 33.1 | | 10.2 | 54.9 |
| RhuIFN_{γ} | 11.6 | 12.2 | 10.2 | | 10.2 |
| rovIFNτ | 48.8 | 33.8 | 54.9 | 10.2 | |

Sequence comparison determined from the following references:
Taniguchi et al., Gene, 10 (1): 11 (1980).
Adolf et al., Biochim. Biophys. Acta, 1089 (2): 167 (1991).
Streuli et al., Science, 209: 1343 (1980).
Imakawa et al., Nature, 330: 377 (1987).

Recombinant ovine IFNτ (rovlFNτ) is 48.8 percent homologous to IFNα_{2b} and 33.8 percent homologous to IFNβ₁. Because of this limited homology between IFNτ and IFNα and between IFNτ and IFNβ, it cannot be predicted whether or not IFNτ would behave in the same manner as IFNα or IFNβ. IFNτ is also reported to have a low receptor binding affinity for type I receptors on human cells (Brod, S., J. Interferon and Cytokine Res., 18: 841 (1999); Alexenko, A. et al., J. Interferon and Cytokine Res., 17: 769 (1997)). Additionally, the fact that IFNτ is a non-endogeneous human protein generates the potential for systemic neutralizing antibody formation when IFNτ is introduced into the human body (Brod, S., J. Interferon and Cytokine Res., 18: 841 (1999).

The 172 amino acid sequence of ovine-IFNτ is set forth, for example, in U.S. Pat. No. 5,958,402, and its homologous bovine-IFNτ sequence is described, for example, in Helmer et al., J. Reprod. Fert., 79: 83-91 (1987) and Imakawa, K. et al., Mol. Endocrinol., 3: 127 (1989). The sequences of ovine-IFNτ and bovine-IFNτ from these references are hereby incorporated by reference. The amino acid sequence of ovine IFNτ is shown herein as SEQ ID NO:2.

1. Isolation of IFN-τ

IFNτ may be isolated from conceptuses collected from pregnant sheep and cultured in vitro in a modified minimum essential medium as described by Godkin, J. D., et al., J. Reprod. Fertil. 65: 141-150 (1982) and Vallet, J. L., et al., Biol. Reprod. 37: 1307 (1987). The IFNτ may be purified from the conceptus cultures by ion exchange chromotography and gel filtration. The homogeneity of isolated IFNτ may be assessed by sodium dodecyl sulfate polyacrylamide gel electrophoresis (Maniatis, T., et al., in MOLECULAR CLONING: A LABORATORY MANUAL, Cold Spring Harbor Laboratory, Cold Spring Harbor, N.Y. (1982); Ausubel, F. M., et al., in CURRENT PROTOCOLS IN MOLECULAR BIOLOGY, John Wiley & Sons, Inc., Media, Pa. (1988)), and determination of protein concentration in purified IFNτ samples may be performed using the bicinchoninic (BCA) assay (Pierce Chemical Co., Rockford, Ill.; Smith, P. K., et al., Anal. Biochem. 150: 76 (1985)).

2. Recombinant Production of IFNτ

Recombinant IFNτ protein may be produced from any selected IFNτ polynucleotide fragment using a suitable expression system, such as bacterial or yeast cells. The isolation of IFNτ nucleotide and polypeptide sequences is described in PCT publication WO194/10313, which is incorporated by reference herein.

To make an IFNτ expression vector, an IFNτ coding sequence (e.g, SEQ ID NOS:1 or 4) is placed in an expression vector, e.g., a bacterial expression vector, and expressed according to standard methods. Examples of suitable vectors include lambda gt11 (Promega, Madison Wis.); pGEX (Smith, P. K. et al., Anal. Biochem. 150: 76 (1985)); pGEMEX (Promega); and pBS (Strategene, La Jolla Calif.) vectors. Other bacterial expression vectors containing suitable promoters, such as the T7 RNA polymerase promoter or the tac promoter, may also be used.

Further, a DNA encoding an IFNτ polypeptide can be cloned into any number of commercially available vectors to generate expression of the polypeptide in the appropriate host system. These systems include the above described bacterial and yeast expression systems as well as the following: bacillus expression (Reilly, P. R. et al., BACULOVIRUS EXPRESSION VECTORS: A LABORATORY MANUAL, (1992); Beames et al., Biotechniques, 11: 378 (1991); Clontech, Palo Alto Calif.); plant cell expression, transgenic plant expression, and expression in mammalian cells (Clontech, Palo Alto Calif.; Gibco-BRL, Gaithersburg Md.). The recombinant polypeptides can be expressed as fusion proteins or as native proteins. A number of features can be engineered into the expression vectors, such as leader sequences which promote the secretion of the expressed sequences into culture medium. The recombinantly produced polypeptides are typically isolated from lysed cells or culture media. Purification can be carried out by methods known in the art including salt fractionation, ion exchange chromatography, and affinity chromatography. Immunoaffinity chromatography can be employed, as described above, using antibodies generated based on the IFNτ polypeptides.

In addition to recombinant methods, IFNτ proteins or polypeptides can be isolated from selected cells by affinity-based methods, such as by using appropriate antibodies. Further, IFNτ peptides (e.g. SEQ ID NOS:2 or 3) may be chemically synthesized using methods known to those skilled in the art.

B. In Vivo Studies

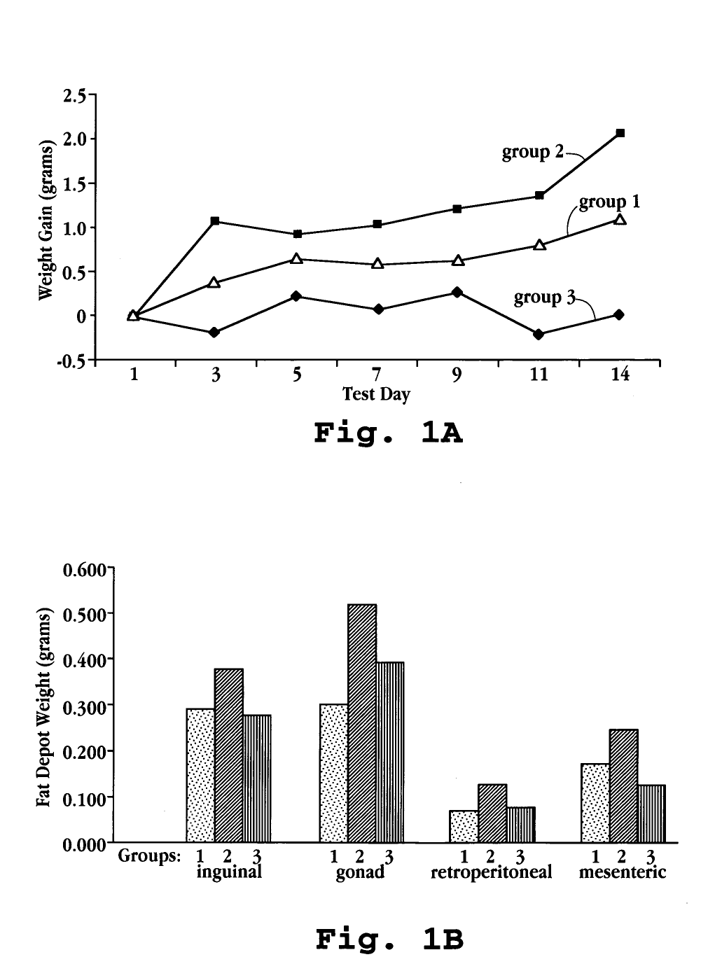

Example 1 describes a study performed on mice fed a high fat diet. Two groups of mice (Groups 2, 3) were provided with a high fat chow for a two week study period. In addition to free access to the chow and water, each mouse in Group 3 was given daily via oral gavage 250 μL of 10 μg IFNτ daily. The mice in Group 3 were given daily via oral gavage 250 μL of buffer. Group 1 animals served as another control, and were fed a normal mouse chow and given buffer via oral gavage daily. The weight of each mouse was monitored over the study period and the results are shown in FIGS. 1A-1B.

FIG. 1A shows the body weight of the mice in each test group over the 14 day study period. The mice in Group 2 that were fed the high fat chow and treated via oral gavage with buffer (squares) had a continual increase in weight over the 14 days. The mice in Group 1 that were fed a conventional mouse chow and treated via oral gavage with buffer (triangles) also had an increase in body weight over the test period, but at a lower rate than the mice fed a high fat diet (Group 2). Mice in Group 3 that were fed a high fat diet and treated with a daily dose of IFNτ (diamonds) had little if any increase in body weight.

On day 14, fat depots from the subcutaneous inguinal, gonadal, retroperitonial, and mesenteric tissues in the test mice were excised and weighed. FIG. 1B shows the average weight of the fat depots for the mice in Group 1 (normal chow, buffer, dotted bars), Group 2 (high fat chow, buffer, cross hatched bars), and Group 3 (high fat chow, IFNτ, vertical striped bars). The mice in Group 2 (high fat chow, buffer) had the highest fat depot weight in all four of the tissues excised. Administration of IFNτ to mice fed a high fat diet resulted in a significant reduction on the deposition of fat, with the fat depots in the inguinal and retroperitoneal areas approximately the same weight as that in mice fed a normal diet (Group 1). The fat depot in the mesenteric tissue for mice treated with IFNτ was less than the control mice in both Groups 1 and 2.

The data in FIGS. 1A-1B indicate that IFNτ is effective as a prophylactic agent to prevent or reduce an increase in body weight in subjects consuming a high fat diet, or in general a diet that provides more calories than needed on a daily basis.

In another study, detailed in Example 2, it was shown that IFNτ is effective as a therapeutic aid in reduction of weight and in weight management. In this study, mice were divided into three treatment groups, identified herein as Groups 4, 5, and 6. The three groups were fed a high fat chow for 23 days and beginning on day 24 were treated daily via oral gavage with a buffer (control, Group 4), 1 μg IFNτ Group 5), or 10 μg IFNτ (Group 6).

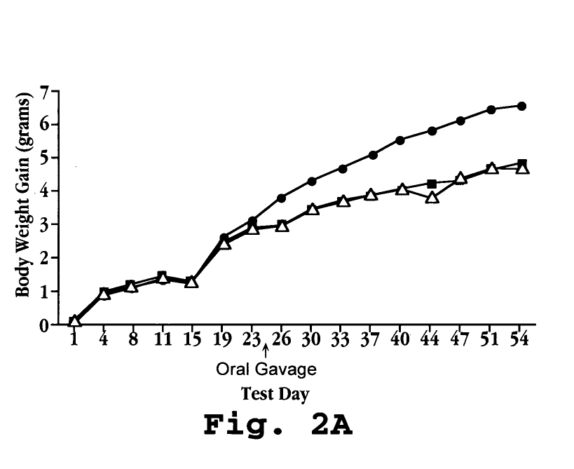

FIG. 2A is a graph showing the average body weight gain of the mice in the test Groups. The weight gain in all mice across the test groups was similar over the first 23 days of the study, prior to administration of the test substances. Over the first 23 days the mice, when fed a high fat diet, gained an average of 3 grams. Beginning on day 24, the mice were treated with buffer (Group 4) or with IFNτ (Groups 5, 6) daily. The difference in continued weight gain beginning on day 24 between Group 4 and Groups 5, 6 is apparent in FIG. 2A. The mice in Group 4 (high fat chow, buffer; circles) continued to gain weight. Mice treated with IFNτ at daily doses of 1 μg (squares, Group 5) or 10 μg (triangles, Group 6) gained less weight than the mice not treated with IFNτ. Table 1 summarizes the average weight gain at various test days for the mice in the test Groups.

TABLE 1

| | Average Weight Gain (grams) | | |
|---|---|---|---|
| Test Day | Group 4¹ | Group 5¹ | Group 6¹ |
| 1 | 0 | 0 | 0 |
| 4 | 0.81 | 1.04 | 0.87 |
| 8 | 1.1 | 1.2 | 1.1 |
| 11 | 1.3 | 1.5 | 1.4 |
| 15 | 1.2 | 1.5 | 1.3 |
| 19 | 2.6 | 2.4 | 2.5 |
| 23 | 3.1 | 2.9 | 2.9 |
| 26 | 3.8 | 3.0 | 2.9 |
| 30 | 4.2 | 3.5 | 3.5 |
| 33 | 4.6 | 3.6 | 3.6 |
| 37 | 5.0 | 3.7 | 3.8 |
| 40 | 5.5 | 3.9 | 4.0 |
| 44 | 5.8 | 4.1 | 3.8 |
| 47 | 6.0 | 4.3 | 4.4 |
| 51 | 6.4 | 4.6 | 4.6 |
| 54 | 6.6 | 4.7 | 4.6 |

¹All test groups fed a high fat diet, with Group 4 and Group 6 treated daily beginning on day 24 with 1 μg IFNτ (Group 5) or 10 μg IFNτ (Group 6)

FIG. 2B presents the body weight results as a bar graph. The dotted bars for each treatment group correspond to the average body weight gain over the 54 day study period. The animals in Group 4 that were fed a high fat diet and treated with buffer as controls had an overall weight increase of 6.6 grams. In contrast, animals treated with IFNτ had a significantly lower overall weight increase of about 4.6 grams, 30% lower than that of the control animals not treated with IFNτ. The bars with cross-hatching correspond to the average weight gain in each treatment group for the period of days 24-54, when IFNτ was administered to test Groups 5 and 6. During this period, the animals treated with IFNτ had an increase in body weight of about 1.8 grams. The animals not treated with IFNτ (Group 4) had an increase in body weight of 3.5 grams, an approximately two-fold higher body weight increase than animals receiving IFNτ. The difference in weight gain between the animals treated with IFNτ and those left untreated was statistically significant (p<0.02). There was no statistical difference between the 1 μg (Group 5) and 10 μg (Group 6) dosages, indicating the minimum therapeutic dose may be lower than 1 μg per day.

The data in FIGS. 2A-2B indicate that IFNτ is effective as a therapeutic agent for management of weight gain and to prevent excessive weight gain when more calories than needed are consumed each day.

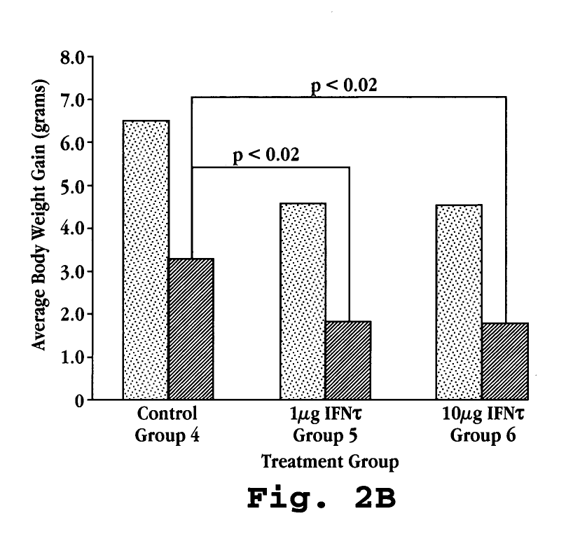

With continuing reference to the study described in Example 2, the animals in Groups 4, 5, and 6 were given an intraperitoneal blood glucose tolerance test on test day 40. After a 4 hour fast, a baseline blood sample was taken and then the animals were given an intraperitoneal injection of glucose. Blood was taken at intervals after glucose administration and analyzed for glucose concentration. The results are shown in FIG. 3, where the blood glucose concentration for animals in the control Group 4 (circles), the test Group 5 treated with 1 μg IFNτ (squares), and the test Group 6 treated with 10 μg IFNτ (triangles) are plotted against time. Animals in all test groups had a sharp increase in blood glucose concentration at the first reading 15 minutes after injection of the glucose. The blood glucose levels then decreased, with the blood glucose levels approaching baseline 90 minutes after glucose administration. This data provides evidence that administration of IFNτ did not result in glucose intolerance in the test animals.

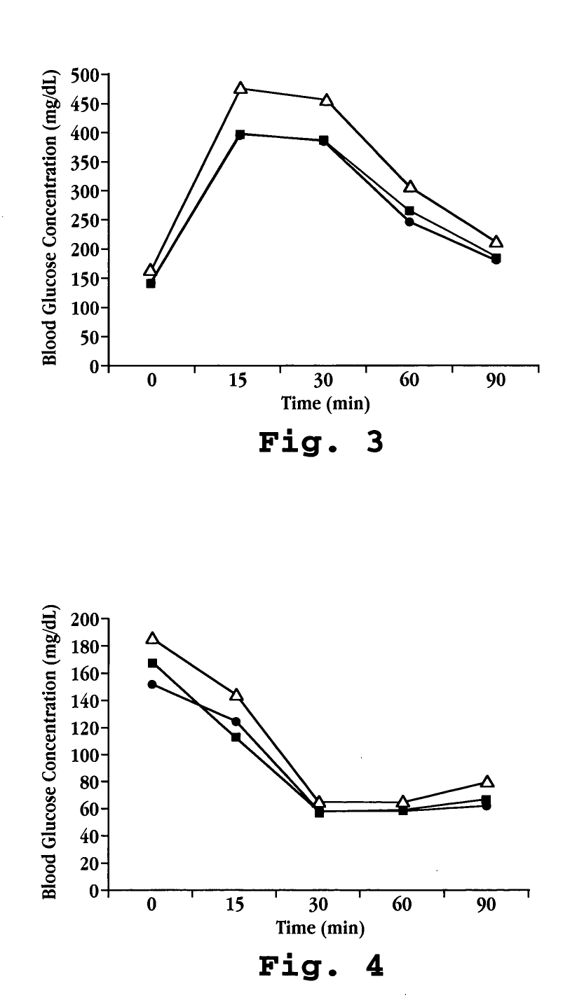

The animals in Groups 4, 5, and 6 were also given an intraperitoneal insulin suppression test on test day 44. After a 4 hour fast, a baseline blood sample was taken and then the animals were given an intraperitoneal injection of insulin. Blood was taken at intervals and analyzed for glucose concentration. The results are shown in FIG. 4, where the blood glucose concentration for animals in the control Group 4 (circles), the test Group 5 treated with 1 μg IFNτ (squares), and the test Group 6 treated with 10 μg IFNτ triangles) are plotted against time after insulin injection. Animals in all test groups had a sharp decrease in blood glucose concentration in the first 30 minutes following insulin administration, with the blood glucose concentration leveling off after 30 minutes. The data indicates that the IFNτ did not adversely effect the absorption of glucose by the tissue, particulary adipose tissue, in the animals.

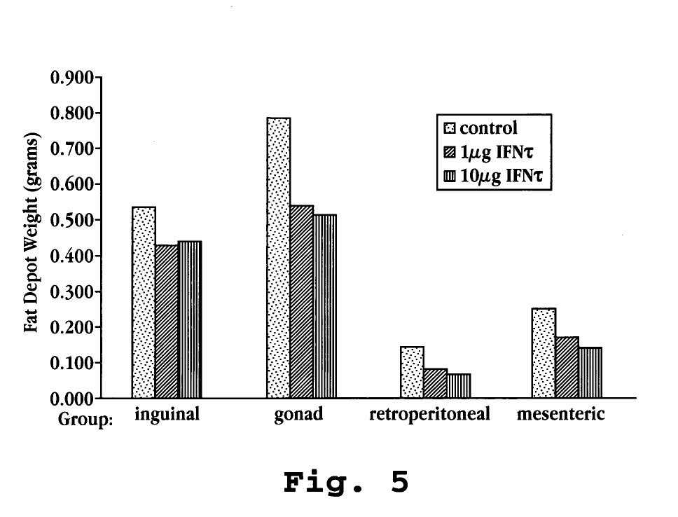

Upon completion of the 54-day study, fat depots were excised from inguinal, gonadal, retroperitoneal, mesenteric regions of the mice. FIG. 5 is a bar graph showing average weight, in grams, of the fat depots for the mice in Group 4 (control, dotted bars), Group 5 (1 μg IFNτ, cross hatched bars), and Group 6 (10 μg IFNτ, vertical stripes). The weight of the fat depots for the control animals fed a high fat diet was greater than for the animals fed a high fat diet and treated therapeutically with IFNτ.

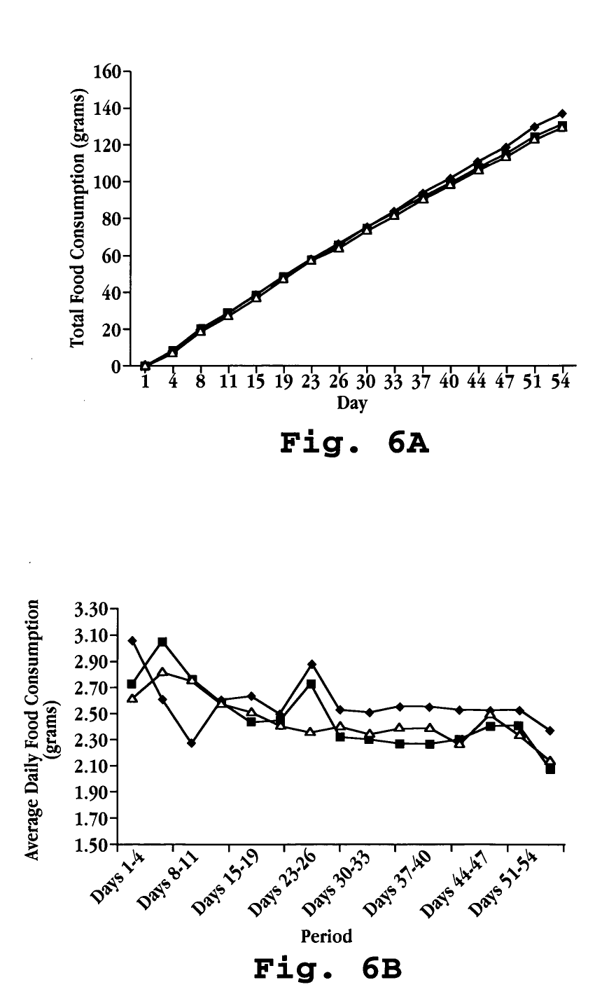

An analysis of the food consumed by the animals in each test group was done to rule out weight gain due to differences in caloric intake. As seen in FIG. 6A, the average, cumulative food consumption by the mice in each treatment group (Group 1 control (circles), Group 2: 1 μg IFNτ (squares), Group 3: 10 μg IFNτ(triangles)) was nearly identical. Thus, the differences in weight gain and weight of fat depots is attributable to the IFNτ rather than a difference in food consumption. FIG. 6B presents the food consumption data as average, daily food consumption for several test periods, defined as about 4 test days. Presented this way, the data shows the control mice (Group 1, circles) consumed less food over the first two test periods (Days 14, Days 8-11) than the mice in the groups treated with IFNτ; however the control mice had an increase in food consumption around the test period of days 23-26.

C. Formulations and Dosages

Accordingly, the studies described in section B above establish that administration of IFNτ to subjects fed a high fat, high sucrose diet gain less weight than subjects not treated with IFNτ. Moreover, overweight or obese subjects treated with IFNτ and consuming a high fat diet gained less weight than subjects not treated with IFNτ. IFNτ was administered to the subjects in the studies via oral gavage; accordingly, the invention contemplates oral administration of IFNτ to those in need of treatment. While oral administration is a preferred route due to ease of administration and improved patient compliance, the method is not limited to oral administration and all possible routes of delivery of IFNτ are included herein.

Oral preparations containing IFNτ can be formulated according to known methods for preparing pharmaceutical compositions. In general, the IFNτ therapeutic compositions are formulated such that an effective amount of the IFNτ is combined with a suitable additive, carrier and/or excipient in order to facilitate effective oral administration of the composition. For example, tablets and capsules containing IFNτ may be prepared by combining IFNτ (e.g., lyophilized IFNτ protein or highly concentrated IFNτ solutions) with additives such as pharmaceutically acceptable carriers (e.g., lactose, corn starch, microcrystalline cellulose, sucrose), binders (e.g., alpha-form starch, methylcellulose, carboxymethylcellulose, hydroxypropylcellulose, hydroxypropylmethylcellulose, polyvinylpyrrolidone), disintegrating agents (e.g., carboxymethylcellulose calcium, starch, low substituted hydroxy-propylcellulose), surfactants (e.g., Tween 80, polyoxyethylene-polyoxypropylene copolymer), antioxidants (e.g., L-cysteine, sodium sulfite, sodium ascorbate), lubricants (e.g., magnesium stearate, talc), or the like.

Further, IFNτ polypeptides of the present invention can be mixed with a solid, pulverulent, or other carrier, for example lactose, saccharose, sorbitol, mannitol, starch, such as potato starch, corn starch, millopectine, cellulose derivative or gelatine, and may also include lubricants, such as magnesium or calcium stearate, or polyethylene glycol waxes compressed to the formation of tablets. By using several layers of the carrier or diluent, tablets operating with slow release can be prepared.

Liquid preparations for oral administration can be made in the form of elixirs, syrups, gels, sprays, or suspensions, for example solutions containing from about 0.1% to about 30% by weight of IFNτ, sugar and a mixture of ethanol, water, glycerol, propylene, glycol and possibly other additives of a conventional nature. Liquid preparations can be administered orally to the stomach and the intestines, or sublingually, or to the oral pharyngeal region, consisting of the base of tongue, the tonsillar region, soft palate, and back of the mouth.

Another suitable formulation is a protective dosage form that protects the protein for survival in the stomach and intestines until absorbed by the intestinal mucosa. Protective dosage forms for proteins are known in the art, and include enteric coatings and/or mucoadhesive polymer coatings. Exemplary mucoadhesive polymer formulations include ethyl cellulose, hydroxypropylmethylcellulose, Eudragit®, carboxyvinyl polymer, carbomer, and the like. A dosage form designed for administration to the stomach via ingestion for delivery of IFNτ in an active form to the intestinal tract, and particularly to the small intestine, is contemplated. Alternatively, IFNτ can be co-administered with protease/peptidase inhibitors, stabilized with polymeric materials, or encapsulated in a lipid or polymer particle to offer some protection from the stomach and/or intestinal environment.

Preparations suitable for administration nasally, bucally, or sublingually are also contemplated. Nasal preparations can be in the form of a liquid suitable for delivery as a spray or mist via the nostrils into the nasal passages and or throat region are readily prepared by those of skill. Preparations in the form of a tablet, capsule, lozenge, or gum that can be held in the mouth for buccal delivery are also readily prepared by those of skill. Sublingual preparations can also take the form of a spray, liquid, gel, powder, tablet, capsule, lozenge or gum.

Preparations suitable for delivery via injection are also contemplated, and any route of injection is suitable, including subcutaneous, intramuscular, intravenous, etc.

The IFNτ can also be admixed with food or drink as a simple means of oral administration. In particular, administration of IFNτ in a snack food or soft drink is a mode of administration that will appeal to a wide patient population. Alternatively, the food or drink can be prepared to contain IFNτ either by mixing the IFNτ with the food or drink or by recombinant production of a food or drink component capable of producing IFNτ. As discussed above, DNA encoding an IFNτ polypeptide can be cloned into any number of commercially available vectors to generate expression of the polypeptide in a plant that is intended for consumption. A fruit or vegetable that is high in IFNτ content can be consumed alone or as part of a prepared food or drink.

The IFNτ pharmaceutical composition is administered in a therapeutically-effective amount to an individual in need of treatment. The dose may vary considerably and is dependent on factors such as the degree of being overweight or obese, seriousness of any secondary disorders, the age and the weight of the patient, other medications that the patient may be taking, and the like. This amount or dosage is typically determined by the attending physician. The dosage will typically be between about 1×10³ and 1×10⁹ units/day, more preferably between about 1×10⁴ and 1×10⁹ units/day, more preferably between about 1×10⁵ and 1×10⁹ units/day. In specific embodiments, IFNτ is administered orally at a dosage of greater than about 1×10³ units/day, preferably of greater than about 1×10⁴ units/day, more preferably of greater than about 1×10⁵ units/day, and still more preferably greater than about 1×10⁶ units/day.

It will be appreciated that a dosage specified in terms of units/day, e.g., 1×10⁴ units/day, refers to antiviral Units of protein per day, where the antiviral activity of IFNτ is measured using a standard cytopathic effect inhibition assay, as described in the literature (Familletti, P. C., et al., Methods in Enzymology, 78: 387-394 (1981); Rubinstein, S. et al., J. Virol., 37: 755-758 (1981)). It will be appreciated that the amount (i.e., mg) of protein to provide a daily dosage of a given number of Units will vary according to the specific antiviral activity of the protein.

The dosage of IFNτ can be administered daily, or can be delivered as often as about every two to four hours if a steady state blood concentration is desired. Less frequent intervals, e.g., once a day or once every 48 hours are also contemplated and may be suitable for some patients. The rate of administration of individual doses is typically adjusted by an attending physician to enable administration of the lowest total dosage while alleviating the severity of the condition(s) being treated. Treatment of the subject at one or more dosages can range from a single dosage given one time, to a dose given more than one time, to an on-going lifetime treatment regimen of the same dose or of variable dosages.

It will, of course, be understood that the administration of IFNτ in accord with the invention may be used in combination with other therapies. For example, IFNτ can be accompanied by administration of other agents suitable for weight management or reduction or in combination with a second therapeutic agent having activity for a secondary condition (i.e., a condition secondary to obesity), such as heart disease, high cholesterol, hypertension, diabetes, arthritis, multiple sclerosis, or psoriasis. More generally, IFNτ may be administered with known immunosuppressants, such as steroids, to treat autoimmune diseases such as multiple sclerosis. The immunosuppressants may act synergistically with IFNτ and result in a more effective treatment that could be obtained with an equivalent dose of IFNτ or the immunosuppressant alone. Co-administration of food supplements to ensure proper nutrition is also contemplated.

III. Exemplary Applications

A. Promote Weight Loss and Prevent Excessive Weight Gain

In a first aspect, the invention provides a method for promoting weight loss in a human subject. In the method, IFNτ is administered in an amount effective to cause a reduction in fat deposition, as measured by a reduction in body weight or by a reduction in the patient's in body mass index. Body mass index (BMI) is a recognized clinical and epidemiological measure for the classification of obesity. The BMI is defined as weight in kilograms divided by the square of height in meters. Typically, a BMI of 25-30 is considered as overweight and greater than 30 as obese. The method of the invention related to promoting weight loss, is preferably directed to those with a BMI of greater than 30, however it will be appreciated that those with a BMI of 25-30 and (i) suffering from a second medical condition that is aggravated by excess weight or (ii) at risk of developing a medical condition due to excess weight will also benefit from the treatment method. Treatment according to the present invention generally refers to a lowering of BMI to less than about 29 to 31 for persons categorized initially as “obese”, or to a BMI of less than about 24-26 for persons initially categorized as “overweight.” It will however be appreciated by persons skilled in the art that obesity is inherently difficult to classify, and that the cut-off point for the definition of obesity is necessarily arbitrary, in part because body fatness is a continuum. However, in general terms, treatment according to the present invention desirably prevents or alleviates obesity to an extent where by there is no longer a significant health risk to the patient.

In another aspect, the invention provides a method for a person to induce weight loss with no change in caloric intake, by administering a composition of IFNτ. As shown above with respect to the study in Example 1, subjects fed a high fat diet (e.g., high calorie diet) in conjunction with IFNτ did not gain excessive weight, whereas subjects consuming the same diet with no IFNτ therapy gained weight. In particular, excessive weight gain can be prevented by administering IFNτ even when the subject consumes a greater than required amount of calories daily, a greater than recommended amount of fat daily and/or a greater than recommended amount of sugar daily, or more generally, a greater than recommended intake of calories from food. It will be appreciated that recommended daily amounts of calories, calories from fat, calories from processed sugar, are based on the age, sex, height, weight, and activity level of an individual. Recommended caloric intake can be found in many reference books or from skilled medical providers or from nutritional experts.

In yet another aspect, the invention provides a method for promoting weight loss in a human subject by administering a composition comprising IFNτ. As shown above with respect to the study described in Example 2, weight loss or prevention of continued weight gain can be induced in overweight or obese subjects by administering IFNτ. Promotion of weight loss typically involves an initial determination of the amount of body weight to be lost. This determination can be based on the desired BMI for the person or on the recommended weight for the persons height and frame size. IFNτ is administered to the person until the desired amount of body weight is lost, at which time the IFNτ therapy can be discontinued, if desired. Alternatively, IFNτ therapy can be continued as a prophylactic measure at the same dosage or at a reduced dosage.

The invention further contemplates a method to prevent weight gain in a subject that is greater than required for normal growth and development. For example, children and young adults gain weight as they grow, and this weight gain is a normal part of growth and development. However, weight gain beyond this expected and required increase is undesirable. A weight gain beyond what is expected for growth and development is readily determined by the height-weight charts used by physicians in monitoring the growth and development of children. A weight gain that results is outside the recommended range for a given height is a weight gain in excess of that required or expected for growth and development. In such patients, IFNτ therapy to prevent excessive weight gain is contemplated.

In another embodiment, the invention contemplates administration of IFNτ in response to an episode of overeating or to an episode of binge eating. Here, a single large dose of IFNτ or multiple smaller doses, after such an episode is contemplated to prevent weight gain due to the consumption of too many calories.

B. Weight Management in Patient Populations with Secondary Condition or at Risk of Developing a Secondary Condition Due to Excess Weight or Fat Deposits

As discussed above in the background section, more than half of U.S. adults are overweight (BMI between 25-30 inclusive of 25) and nearly one-quarter of the U.S. adults are considered to be obese (BMI equal to or greater than 30). The increasing prevalence of overweight and obesity is a major public health concern, since obesity is associated with several chronic diseases. For example, overweight and obesity are known risk factors for diabetes, heart disease, stroke, hypertension, gallbladder disease, osteoarthritis, sleep apnea, and some forms of cancer such as uterine, breast, colorectal, kidney, and gallbladder. Furthermore, obesity is associated with high cholesterol, complications of pregnancy, menstrual irregularities, hirsutism, and increased surgical risk.

Accordingly, in another aspect the invention contemplates methods for treating a patient population (1) at risk of developing a medical condition due to a state of being overweight or obese or (2) suffering from a condition where the symptoms or the condition itself are exacerbated by excess weight or (3) suffering from a condition that causes risk of becoming overweight or obese due to actual or perceived impaired physical ability. Based on the studies discussed above, it is readily appreciated that delivery of IFNτ to such patient populations will result in a prevention of further weight gain and/or a reduction in body weight, both of which will favorably impact an actual or threatened second medical condition. In a preferred aspect, the IFNτ offers a therapeutic benefit to the actual or threatened second medical condition in these various patient populations, as will now be discussed.

IFNτ has been shown to have a therapeutic benefit in the treatment of viral diseases, cellular proliferation diseases, as will as autoimmune disorders. In a preferred embodiment, IFNτ is administered to patients having a BMI greater than about 25 and at risk of developing or already suffering from an autoimmune disoreder. Autoimmune disorders may be loosely grouped into those primarily restricted to specific organs or tissues and those that affect the entire body. Examples of organ-specific disorders (with the organ affected) include multiple sclerosis (myelin coating on nerve processes), type I diabetes mellitus (pancreas), Hashimotos thyroiditis (thyroid gland), pernicious anemia (stomach), Addison's disease (adrenal glands), myasthenia gravis (acetylcholine receptors at neuromuscular junction), rheumatoid arthritis (joint lining), uveitis (eye), psoriasis (skin), Guillain-Barré Syndrome (nerve cells) and Grave's disease (thyroid). Systemic autoimmune diseases include systemic lupus erythematosus and dermatomyositis.

Autoimmune diseases particularly amenable for treatment using the methods of the present invention include diabetes mellitus, lupus erythematosus, rheumatoid arthritis, multiple sclerosis, and psoriasis. Diabetes is a chronic metabolic disorder which afflicts 16 million people in the United States, over one and one half million of whom have its most severe form, childhood diabetes (also called juvenile, type 1 or insulin-dependent diabetes). Insulin-dependent diabetes appears suddenly, most often in children and young adults, and progresses rapidly. In this form, the pancreas ceases to manufacture insulin, a hormone necessary to convert food into energy for the body. Virtually every major organ system in the body is damaged by diabetes. Complications can include blindness, kidney failure, heart disease, stroke, amputation of extremities, loss of nerve sensation, early loss of teeth, high-risk pregnancies and babies born with birth defects. Currently, insulin injection is the treatment method of choice for type 1 diabetics and becomes the eventual course of treatment for many of the type 2 diabetics in the United States. Other autoimmune conditions include Graves disease and ankylosing spondylitits.

Excessive weight, e.g., persons categorized as overweight (BMI of 25 or greater and less than 30) or obese (BMI of 30 or greater) is one of the causative factors of life-style leading to diabetes. Accordingly, a method for managing weight, e.g., promoting weight loss or preventing weight gain, is beneficial to those at risk of diabetes or suffering from diabetes. Administration of IFNτ to manage weight in such a patient population is desirable. Further, because of the therapeutic effect of IFNτ on autoimmune disorders, a supplemental therapeutic benefit is achieved.

Arthritis involves inflammation of a joint and is characterized by pain, swelling, stiffness, and redness. Rheumatoid arthritis is one type of arthritis, and is the most severe type of inflammatory joint disease. Rheumatoid arthritis is an autoimmune disorder where the body's immune system acts against and damages joint and surrounding soft tissues. The joints, and particularly those in the hands, feet, and arms, become extremely painful, stiff, and deformed. Patients suffering from rheumatoid arthritis experience a worsening of symptoms and often have a poorer prognosis when carrying excessive weight. Additionally, the conditions makes movement painful, thus limiting physical activity which can lead to weight gain. The method of the invention contemplates administering IFNτ to one or more persons in a patient population comprised of persons suffering from rheumatoid arthritis and (i) having a BMI of greater than about 25, more preferably greater than about 30, and/or (ii) experiencing an increasing weight or BMI due to restriction of physical abilities. Treatment with IFNτ can promote weight loss and/or prevent further weight gain, thereby alleviating the aggravation of the arthritic condition due to excessive weight.

Lupus erythematosus is a chronic disease that causes inflammation of connective tissue. Discoid lupus erythematosus is the more common type and affects exposed areas of the of the skin. Systemic lupus erythematosus is a more serious and potentially fatal form, affecting may systems of the body including the joints and kidneys. Both forms of lupus are autoimmune disorders where the body's immune system attacks the connective tissue causing inflammation. Lupus patients can experience a restricted physical mobility, due to joint tenderness and inflammation. Limited physical activity can lead to weight gain. The method of the invention contemplates administering IFNτ to one or more persons in a patient population comprised of persons suffering from lupus erythematosus and (i) having a BMI of greater than about 25, more preferably greater than about 30, and/or (ii) experiencing an increasing weight or BMI due to restriction of physical abilities. Treatment with IFNτ can promote weight loss and/or prevent further weight gain, thereby alleviating the aggravation of the joint tenderness due to excessive weight.

Multiple sclerosis is a progressive disease of the central nervous system where scattered patches of myelin, the protective covering of nerve fibers, in the brain and spinal cord are destroyed. This causes symptoms ranging from numbness and tingling to paralysis and incontinence. The subject's extremities may feel heavy and become weak. Multiple sclerosis is an autoimmune disease in which the body's defense system begins to treat the myelin as foreign, gradually destroying it, with subsequent scarring and damage. MS patients can experience a restricted physical mobility, due to the symptoms described above. Being overweight or obese compounds the symptoms, and limited physical activity due to the symptoms can lead to weight gain. The method of the invention contemplates administering IFNτ to a patient population comprised of persons suffering from multiple sclerosis and (i) having a BMI of greater than about 25, more preferably greater than about 30, and/or (ii) experiencing an increasing weight or BMI due to restriction of physical abilities. Treatment with IFNτ can promote weight loss and/or prevent further weight gain, thereby alleviating the aggravation of MS symptoms due to excessive weight.

Psoriasis is an autoimmune skin disease characterized by thickened patches of inflamed, red skin, often covered by silvery scales. Regions of skin affected by psoriasis may be so extensive that great physical discomfort and social embarrassment result. Areas of skin eruption can be accompanied by painful swelling and stiffness of joints that can be highly disabling. Suffers of psoriasis can experience a restricted physical mobility, due to the joint pain and or due to a perceived inability to participate in exercise. Being overweight or obese compounds the joint, and limited physical activity due to the symptoms can lead to weight gain. The method of the invention contemplates administering IFNτ to one or more persons in a patient population comprised of those suffering from psoriasis and (i) having a BMI of greater than about 25, more preferably greater than about 30, and/or (ii) experiencing an increasing weight or BMI due to restriction of physical abilities. Treatment with IFNτ can promote weight loss and/or prevent further weight gain, thereby alleviating the aggravation of psoriasis symptoms due to excessive weight.

Cardiovascular disease is another common disorder leading to health problems. In another aspect, the invention provides for a method of treating one or more persons in a patient population having a BMI of greater than about 25, more preferably greater than about 30, and additionally suffering from a cardio-vascular disorder. Such a patient population can be those at risk of stroke, high blood pressure, or high cholesterol. Alternatively, the patient population can be persons who have suffered a stroke or who currently have high blood pressure or high cholesterol. In a patient population currently suffering from a cardio-vascular related disorder, IFNτ is administered to alleviate the symptoms of the cardio-vascular disorder that are exacerbated by the excessive weight, i.e., IFNτ is administered to promote weight loss or to prevent further weight gain. In this aspect, co-administration of IFNτ with known therapeutic agents for treatment of high blood pressure, high cholesterol, heart disease is contemplated.

### IV. EXAMPLES

The following examples further illustrate the invention described herein and are in no way intended to limit the scope of the invention.

Materials: Interferon-tau was prepared according to known techniques previously described (see, for example, U.S. 2004/0191217). The IFNτ had a specific activity of about 1×10⁸ antiviral U/mg protein.

### Example 1

### In Vivo Administration of IFNτ Prophylacticaliy

Eighteen C57 Black mice (The Jackson Laboratory, Bar Harbor Me.), housed individually in a 12:12 light:dark cycle, were randomly divided into 3 test groups. The test animals had free access to food and water during the study, with the food provided to the groups as follows:

| Group No. | |
|---|---|
| (n = 6) | Diet |
| 1 | normal chow, buffer |
| 2 | high-fat chow, buffer |
| 3 | high-fat chow, 10 μg IFNτ |

The high fat chow was a high fat chow (60% kCal, Research Diets, Inc. New Brunswick, N.J.). In addition to the chow, each mouse was treated daily via oral gavage with 250 μL of buffer (Groups 1, 2) or 10 μg IFNτ (Group 3). The body weight of the animals was monitored over a 14 day period, and the results are shown in FIGS. 1A-1B.

### Example 2

### In Vivo Administration of IFNτ Therapeutically

Twenty-one male specific pathogen free (SPF) mice (6 weeks old, C57B1.6J, The Jackson Laboratory, Bar Harbor Me.) were randomly divided into 3 test groups. The mice were acclimated for 5 days prior study initiation. The animals were housed individually in a 12:12 light:dark cycle. The mice had free access to food and water, the food provided was a high fat (60% kCal, Research Diets, Inc. New Brunswick N.J.), high sucrose diet (Research Diet).

After 23 days on the high fat/high sucrose diet the animals in each test group were treated daily as follows:

| Group No. | Treatment on Days |
|---|---|
| (n = 7) | 24-27 |
| 4 | saline (control) |
| 5 | 1 μg IFNτ |
| 6 | 10 μg IFNτ |

The IFNτ dose or the saline control were administered daily by oral gavage of 250 μL to each mouse in the test group. The animals were observed daily for the duration of the study and body weights were taken on days 1, 4, 8, 11, 15, 19, 23, 26, 30, 33, 37, 40, 44, and 47. The amount of food consumed by each mouse was monitored, and the weight of food ingested determined on the same test days that body weights were determined.

1. Intraperitoneal Glucose Tolerance (IPGTT) Test

On test day 40, the animals were fasted for 4 hours. An intraperitoneal glucose tolerance (IPGTT) test was performed. A fasting blood sample was taken from the tail vein and a concentrated solution of glucose (2 g/kg body weight) was injected into the abdominal cavity of each mouse through a needle passed through the abdominal skin. Blood samples were removed from the tail vein at 15, 30, 60 and 90 minutes for analysis of glucose and insulin concentrations. The data is shown in the table below and plotted in FIG. 3.

| | Blood Glucose Concentration (mg/dL) | | |
|---|---|---|---|
| Time | Group 4 | Group 5 | Group 6 |
| 0 | 141 | 142 | 165 |
| 15 | 394 | 397 | 476 |
| 30 | 36 | 382 | 456 |
| 60 | 246 | 262 | 307 |
| 90 | 178 | 183 | 210 |

2. Dexa Scans

On test day 43, Dexa scans (Norland Instruments Dual X-Ray) were taken to determine total body fat and total lean tissue mass.

3. Intraperitoneal Insulin Suppression Test (IPIST)

On test day 44, the animals were fasted for 4 hours. An intraperitoneal insulin suppression test (IPIST) was performed. A fasting blood sample was taken from the tail vein and insulin (0.75 units/kg body weight) was injected into the abdominal cavity of each mouse through a needle passed through the abdominal skin. Blood samples were removed from the tail vein 10, 30, and 60 minutes for glucose clearance measurements. The results are shown in the table below and in FIG. 4.

| | Blood Glucose Concentration (mg/dL) | | |
|---|---|---|---|
| Time | Group 4 | Group 5 | Group 6 |
| 0 | 151 | 166 | 186 |
| 10 | 124 | 113 | 144 |
| 30 | 59 | 56 | 64 |
| 60 | 56 | 58 | 64 |
| 90 | 66 | 62 | 79 |

4. Lipid Profile Determination. Fat Depot Excision

On test day 47, the mice were sacrificed. Serum was collected and stored for lipid profile analysis of total cholesterol, high density lipids, and low density lipids. The fat depots were excised from the inguinal, gonadal, retroperiotneal and mesenteric areas.

Although the invention has been described with respect to particular embodiments, it will be apparent to those skilled in the art that various changes and modifications can be made without departing from the invention.

1. A method for promoting weight loss in a human, comprising
administering to said human a composition comprising an amount of interferon-tau effective to achieve a reduction in fat deposition, as measured by a reduction in body weight.

2. The method of claim 1, wherein said administering is to a human in need of weight loss.

3. The method of claim 1, wherein said administering is to a human consuming greater than the daily recommend fat intake for a human of that age, sex, and weight.

4. The method of claim 1, wherein said administering is for a period of greater than one day.

5. The method of claim 1, wherein said administering is selected from the group consisting of nasally administering, bucally administering, sublingually administering, and orally administering.

6. The method of claim 5, wherein said administering comprises administering a preparation selected from the group consisting of spray, liquid, gel, powder, tablet, capsule, lozenge, and gum.

7. The method of claim 5, wherein said administering comprises orally administering an amount of interferon-tau admixed with food or drink.

8. The method of claim 1, wherein said administering comprises administering an amount of interferon-tau that is greater than about 10,000 units/day.

9. The method of claim 1, wherein said administering comprises administering a ruminant interferon-tau.

10. The method of claim 9, wherein said administering comprises administering a ruminant interferon-tau having a sequence corresponding to the interferon-tau sequence of a ruminant selected from the group consisting of Ovine, Bovine, Ovibos, Giraffa, Equus, Hippopotamus, Loxodonta, Llama, Capra, and Cervidae.

11. The method of claim 8, wherein said administering comprises administering ovine interferon-tau having a sequence identified as SEQ ID NO:2 or SEQ ID NO:3.

12. The method of claim 1, wherein said method further comprises co-administering a second therapeutic agent.

13. The method of claim 1, wherein said method further comprises co-administering a food supplement.

14. A method for preventing weight gain in a human subject, comprising
administering to said human a composition comprising an amount of interferon-tau effective to prevent weight gain during consumption of one or more of (i) a greater than recommended daily intake of calories for said subject; (ii) a greater than recommended daily intake of fat for said subject; and (iii) a greater than recommend intake of calories from fat for said subject.

15. The method of claim 14, wherein said administering is for a period of time in the range of one day to the subject's lifetime.

16. The method of claim 14, wherein said administering is selected from the group consisting of nasally administering, bucally administering, sublingually administering, and orally administering.

17. The method of claim 16, wherein said administering comprises administering a preparation selected from the group consisting of spray, liquid, gel, powder, tablet, capsule, lozenge, and gum.

18. The method of claim 16, wherein said administering comprises orally administering an amount of interferon-tau admixed with food or drink.

19. The method of claim 14, wherein said administering comprises administering an amount of interferon-tau that is greater than about 10,000 units/day.

20. The method of claim 14, wherein said administering comprises administering a ruminant interferon-tau.

21. The method of claim 20, wherein said ruminant interferon-tau has a sequence corresponding to the interferon-tau sequence of a ruminant selected from the group consisting of Ovine, Bovine, Ovibos, Giraffa, Equus, Hippopotamus, Loxodonta, Llama, Capra, and Cervidae.

22. The method of claim 20, wherein said interferon-tau is ovine interferon-tau having a sequence identified as SEQ ID NO:2 or SEQ ID NO:3.

23. The method of claim 14, wherein said method further includes co-administering a second therapeutic agent.

24. The method of claim 14, wherein said method further includes co-administering a food supplement.

25. A method for promoting weight loss in a human, comprising
determining a desired amount of body weight to be lost;
administering to said human a composition comprising an amount of interferon-tau; and
discontinuing said administering when the desired amount of body weight is lost.

26. The method of claim 25, wherein said method further comprises maintaining the daily caloric intake of the human at a level equal to or greater than that prior to said administering.

27. The method of claim 25, wherein said administering is selected from the group consisting of nasally administering, bucally administering, sublingually administering, and orally administering.

28. The method of claim 27, wherein said administering comprises administering a preparation selected from the group consisting of spray, liquid, gel, powder, tablet, capsule, lozenge, and gum.

29. The method of claim 27, wherein said administering comprises orally administering an amount of interferon-tau admixed with food or drink.

30. The method of claim 25, wherein said administering comprises administering an amount of interferon-tau that is greater than about 10,000 units/day.

31. The method of claim 25, wherein said administering comprises administering a ruminant interferon-tau.

32. The method of claim 31, wherein said ruminant interferon-tau has a sequence corresponding to the interferon-tau sequence of a ruminant selected from the group consisting of Ovine, Bovine, Ovibos, Giraffa, Equus, Hippopotamus, Loxodonta, Llama, Capra, and Cervidae.

33. The method of claim 31, wherein said interferon-tau is ovine interferon-tau having a sequence identified as SEQ ID NO:2 or SEQ ID NO:3.

34. The method of claim 25, wherein said method further includes co-administering a second therapeutic agent.

35. The method of claim 25, wherein said method further includes co-administering a food supplement.

36. A method for a human to self-induce weight loss with no change in caloric intake, comprising
self-administering a composition comprising an amount of interferon-tau.

37. The method of claim 36 wherein said self-administering comprises self-administration via a route of administration selected from the group consisting of nasal, bucal, sublingual, and oral.

38. The method of claim 37, wherein said self-administering comprises self-administering a preparation selected from the group consisting of spray, liquid, gel, powder, tablet, capsule, lozenge, and gum.

39. The method of claim 37, wherein said self-administering comprises orally administering amount of interferon-tau admixed with food or drink.

40. The method of claim 36, wherein said self-administering comprises administering an amount of interferon-tau is greater than about 10,000 units/day.

41. The method of claim 36, wherein said self-administering comprises administering an interferon-tau that corresponds to an interferon-tau sequence from a ruminant.

42. The method of claim 41, wherein said ruminant interferon-tau has a sequence corresponding to the interferon-tau sequence of a ruminant selected from the group consisting of Ovine, Bovine, Ovibos, Giraffa, Equus, Hippopotamus, Loxodonta, Llama, Capra, and Cervidae.

43. The method of claim 36, wherein said self-administering comprises administering ovine interferon-tau having a sequence identified as SEQ ID NO:2 or SEQ ID NO:3.

44. The method of claim 36, wherein said method further includes co-administering a second therapeutic agent.

45. The method of claim 36, wherein said method further includes co-administering a food supplement.

46. A method for treating a patient population having a body mass index of greater than about 25, and wherein said patient population is at risk of developing a medical condition caused by or related to a higher than desired body mass index, comprising
administering to a patient in said population an amount of interferon-tau effective to promote weight loss and to reduce risk of developing said second medical condition.

47. The method according to claim 46, wherein said administering is via oral administration.

48. The method according to claim 46, wherein said medical condition is selected is type II diabetes.

49. A method for treating a person suffering from an autoimmune disorder and having a body mass index of greater than about 25, comprising
administering to the person an amount of interferon-tau effective to alleviate symptoms associated with said autoimmune disorder and to promote weight loss.

50. The method according to claim 49, wherein said administering is via oral administration or via injection.

51. The method according to claim 49, wherein said autoimmune condition is selected from the group consisting of rheumatoid arthritis, diabetes, multiple sclerosis, lupus, and psoriasis.

52. A method for treating a person having a body mass index of greater than about 25 and suffering from a cardiovascular disorder, comprising
administering to the person an amount of interferon-tau effective to alleviate symptoms associated with said cardiovascular disorder and to promote weight loss.

53. The method of claim 52, wherein said administering is via oral administration.

54. The method of claim 52, wherein said administering is via injection.

55. The method of claim 52, wherein said cardio-vascular disorder is selected from the group consisting of stroke, hypertension, and high cholesterol.

56. The method of claim 52, further comprising co-administering a second therapeutic agent.


SEQUENCE LISTING (4 sequences)

SEQ ID NO: 1 - DNA, 516 bp, Ovis Aries
tgctacctgtcgcgaaaactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaggttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg

SEQ ID NO: 2 - protein, 172 aa, Ovis Aries
CYLSRKLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 3 - protein, 172 aa, Artificial
  Recombinant IFNTau Based on Ovis Aries Sequence
CYLSERLMLDARENLKLLDRMNRLSPHSCLQDRKDFGLPQEMVEGDQLQKDQAFPVLYEMLQQSFNLFYTEHSSAAWDTTLLEQLCTGLQQQLDHLDTCRGQVMGEEDSELGNMDPIVTVKKYFQGIYDYLQEKGYSDCAWEIVRVEMMRALTVSTTLQKRLTKMGGDLNSP

SEQ ID NO: 4 - DNA, 516 bp, Artificial
  Recombinant IFNTau Based on Ovis Aries Sequence
tgctacctgtcggagcgactgatgctggacgctcgagaaaatttaaaactgctggaccgtatgaatcgattgtctccgcacagctgcctgcaagaccggaaagacttcggtctgccgcaggaaatggttgaaggtgaccaactgcaaaaagaccaagctttcccggtactgtatgaaatgctgcagcagtctttcaacctgttctacactgaacattcttcggccgcttgggacactactcttctagaacaactgtgcactggtctgcaacagcaactggaccatctggacacttgccgtggccaagttatgggtgaagaagactctgaactgggtaacatggatccgatcgttactgttaaaaaatatttccagggtatctacgactacctgcaggaaaaaggttactctgactgcgcttgggaaatcgtacgcgttgaaatgatgcgggccctgactgtgtcgactactctgcaaaaacggttaactaaaatgggtggtgacctgaattctccg